(19) United States
(12) Patent Application Publication
(10) Pub. No.: US 2026/0158076 A1
(43) Pub. Date: Jun. 11, 2026

(54) HYDROGEL AND USES THEREOF

(71) Applicants: THE UNIVERSITY OF MELBOURNE, Melbourne (AU); AUSTRALIAN NATIONAL UNIVERSITY, Acton (AU); FLOREY INSTITUTE OF NEUROSCIENCE AND MENTAL HEALTH, Melbourne (AU); DEAKIN UNIVERSITY, Geelong (AU)

(72) Inventors: David Russell NISBET, Melbourne (AU); Colin John JACKSON, Acton (AU); Clare Louise PARISH, Melbourne (AU); Richard James WILLIAMS, Geelong (AU)

(21) Appl. No.: 18/860,968

(22) PCT Filed: Apr. 28, 2023
(86) PCT No.: PCT/AU2023/050352

§ 371 Date: Oct. 28, 2024

(30) Foreign Application Priority Data
Apr. 29, 2022 (AU) 2022901134

(51) Int. Cl. A61K 35/545; A61K 9/08; A61K 47/42; A61K 47/69

(52) U.S. Cl. CPC A61K 35/545; A61K 9/08; A61K 47/42; A61K 47/6903

(57) ABSTRACT

The present invention relates to biocompatible hydrogels, in particular, biocompatible hydrogels for delivering a cell to a subject, wherein the hydrogel comprising a scaffold and an oxygen carrier.

### CROSS-REFERENCE TO RELATED APPLICATIONS

This application claim priority from Australian Patent Application No. 2022901134, the entire contents of which are incorporated herein by reference.

### TECHNICAL FIELD

The present invention relates generally to biocompatible hydrogels, in particular to biocompatible hydrogels for delivering a cell to a subject.

### BACKGROUND

Regenerative medicine has emerged as a significant approach to treating diseases or disorders, including neurodegenerative diseases and disorders. For example, cell transplantation for the treatment of Parkinson's disease (PD) has had some success in numerous clinical trials. However, while in some instances transplanted neurons have been shown to be capable of delivering long-term symptomatic relief for patients, poor reliability and unpredictable and unacceptable variability in functional improvement between patients has hindered its translation to the clinic.

Neurodegeneration associated with PD is a multisystem process. However, the characteristic movement anomalies associated with PD are caused by the accelerated and progressive death of ventral midbrain dopamine neurons, and subsequently the loss of the nigrostriatal pathway connecting the substantia nigra pars compacta with the dorsal striatum.

To date, pharmacological therapy to increase dopamine levels within the brain is the only clinical treatment to offer symptomatic relief to patients suffering PD. However, that approach is far from ideal, with drug administration having little to no impact on disease progression, being burdened by many side effects, and having highly variable efficacy over time. In contrast, cell transplantation has the potential to offer permanent symptomatic relief for PD patients.

The repair and reconstruction of neural circuitry and symptomatic relief of PD has been demonstrated in pre-clinical and clinical trials. Although such studies have been encouraging, significant variability in transplanted cell viability and functional recovery has been reported between patients, associated with variability in cell survival and innervation within the host tissue. This has been attributed, at least in part, to a lack of a supportive regenerative milieu arising from the unpredictable injury environment. Such inconsistent outcomes in preclinical and clinical trials have highlighted a need to advance cell administration strategies to improve therapeutic outcomes following administration of cells within biological tissue, including the brain (i.e., brain tissue).

Injectable hydrogels have previously shown potential for use in regenerative medicine as cellular delivery vectors. However, as with other cell-laden transplantable materials, the administration of cells with injectable hydrogels typically suffers from poor cell survival, differentiation, and functional integration.

Accordingly, there is an ongoing need for improved or alternative methods for delivering a cell to a subject in need thereof, such as transplanted stem cells.

### SUMMARY

The present invention is predicated, at least in part, on the inventors' surprising discovery that hydrogels comprising an oxygen carrier non-covalently bound to the hydrogel scaffold unexpectedly provide sustained presentation of oxygen to a cell, thereby promoting survival of the cell.

Thus, in one aspect, the present invention provides a biocompatible hydrogel for delivering a cell into a subject, the hydrogel comprising a scaffold and an oxygen carrier non-covalently bound to the scaffold.

In another aspect, the present invention provides an injectable self-assembled biocompatible hydrogel for delivering a cell into a subject, the injectable self-assembled hydrogel comprising an IKVAV-based scaffold and myoglobin or a functional variant thereof non-covalently bound to the scaffold.

In another aspect, the present invention provides a method for preparing a biocompatible hydrogel of the invention, comprising:
a) combining a scaffold material and water to form a hydrogel scaffold;
b) combining the hydrogel scaffold with an oxygen carrier to form a homogenous mixture;
c) gelation of the homogenous mixture to form a biocompatible hydrogel in which the oxygen carrier is non-covalently bound to the hydrogel scaffold.

In another aspect, the present invention provides a method for preparing a biocompatible hydrogel of the invention, comprising:
a) combining a scaffold material, an oxygen carrier and water to form a homogenous mixture;
b) gelation of the homogenous mixture to form a hydrogel in which the oxygen carrier is non-covalently bound to the hydrogel scaffold.

In another aspect, the present invention provides use of a biocompatible hydrogel of the invention for delivering a cell into a subject.

In another aspect, the present invention provides a method for transplanting a cell into a subject, the method comprising a subject comprising administering the biocompatible hydrogel of the invention to the tissue.

In yet another aspect, the present invention provides use of a biocompatible hydrogel of the invention in the manufacture of a medicament for transplanting a cell into a tissue of a subject t.

### BRIEF DESCRIPTION OF THE DRAWINGS

Embodiments of the invention will now be described with reference to the following Figures, which are intended to be exemplary only, and in which:

FIG. 1. Characterization of self-assembled peptide (SAP) and SAP: myoglobin hydrogels. (A) Rheological analysis showing characteristic viscoelastic behaviour; (B) Photograph of SAPs alone (left), oxidized myoglobin+SAPs (middle), and reduced myoglolin+SAPs (right).

FIG. 2. Biophysical and biochemical characterization of hydrogels. (A) Rheology data of various hydrogel: myoglobin preparations showing the storage modulus (solid dots) and loss modulus (empty dots) in solid dots with a slight change in stiffness; (B) Circular dichroism absorbance spectra of the hydrogel groups showing the proteins all retain similar secondary structure; (C) FTIR spectra of the hydrogel groups showing the secondary structure containing predominantly β-sheets are formed; (E-I) Representative TEM images of the hydrogel and Mbs hydrogels, coloured according to the legend (D).

FIG. 3. SAXS analysis of SAP (blue) and SAP: myoglobin (red), with curves offset for clarity. The scattering of myoglobin in solution (from the SAS database standard curve, https://www.sasbdb.org/data/SASDAH2/) is shown to indicate where its scattering would be significant.

FIG. 4. UV-vis spectroscopy functional analysis of reduced deoxymyoglobin over a 10 hour period.

FIG. 5. Density of proinflammatory GFAP+astrocytes immediately adjacent to the site of hydrogel administration. (A) Density of GFAP+ reactive astrocytes surrounding the GFP⁺ graft. (B&C): Representative images of GFAP+ immunolabeling adjacent to GFP+ graft in SAPs and SAPs+Myoglobin groups, respectively. Data represents mean±standard error of the mean (SEM).

FIG. 6. Quantification of NeuN+ cell density within a graft 28 days post transplantation. (A) Volume of graft core; (B) Volume of innervation; (C) Representative photomicrographs providing a coronal view of GFP+ graft in SAPs and SAPs+Myoglobin groups including different oxygen affinities, respectively. Data represents mean±standard error of the mean (SEM), (*, p<0.05).

FIG. 7. Volumetric analysis of the GFP+ fibres within surrounding brain parenchyma assessed at 28 days post administration. (A) Volume of innervation; (B&C) Representative photomicrographs providing innervation of GFP⁺ graft in SAPs and SAPs+Myoglobin groups; (D) Density of GFP fibre; (E&E′) present the GFP fibre high magnification in SAPs group; (F&F′) present the GFP fibre high magnification in SAPs+Myoglobin group. Data represents mean±standard error of the mean (SEM).

FIG. 8. Analysis of cell proliferation at 28 days post administration. (A) Density of Ki67⁺ proliferative cells in the graft; (B&C) Representative images of Ki67⁺ immunolabeling in the graft in SAPs and SAPs+Myoglobin groups, respectively; (D) Density of DCX⁺ migrating cells in the graft; (E&F) Representative images of DCX⁺ immunolabeling in the graft in SAPs and SAPs+Myoglobin groups, respectively. Data represents mean±standard error of the mean (SEM). Scale bar represents 50 μm.

FIG. 9. Myoglobin variants. (A) The crystal structure of the Leu29Phe mutant of Physeter macrocephalus myoglobin (High affinity whale Mb, PDB ID: 2SPL), showing that Phe29 stabilizes His64 in a conformation where it can coordinate the bound ligand (in this case CO); (B) The structure of wild-type Physeter macrocephalus myoglobin (Sperm whale Mb, PDB ID: 1VXC); (C) The crystal structure of the His64Leu mutant of Physeter macrocephalus myoglobin (Low affinity whale Mb, PDB ID: 2MGE), showing that the Leu64 mutation removes the ligand-coordinating imidazole sidechain of His64; (D) The electrostatic surface of Physeter macrocephalus myoglobin (Sperm whale Mb); (E) and Equus caballas myoglobin (Horse Mb) (1AZI), showing the greater cationic (blue;circled) surface charge and pI of Physeter macrocephalus myoglobin.

FIG. 10. (a) Sequence alignment between Physeter macrocephalus (sperm whale) myoglobin (MYGPHYMC) and Equus caballus (horse) myoglobin (MYGHORSE). (b) Location of the sequence differences between Physeter macrocephalus and Equus caballus myoglobin.

FIG. 11. Stability of (A) horse and (B) human wild type Mb at 37° C. The absorbance reading was performed on a CLARIOSTAR (BMG LABTECH) plate reader measuring absorbance spectrums (scan resolution 1 nm, 300-800 nm) every 10 min up to 2 hrs. The test was performed at controlled temperature and CO₂ conditions using an atmospheric control unit (BMG LABTECH), setting 37 C and 5% CO₂. Y-axis shows the fold-change in optical density at 410 nm, blank corrected for PBS or peptides alone, and were normalized by time 0 and plotted as mean and standard deviation. Statistical analysis was performed using a two-way ANOVA (p>0.05).

### DEFINITIONS

Unless defined otherwise, all technical and scientific terms used herein have the same meanings as commonly understood by one of ordinary skill in the art to which the present disclosure belongs. Any materials and methods similar or equivalent to those described herein can be used to practice the present invention.

As used herein, the term “conservative amino acid substitution” refers to changing amino acid identity at a given position to replace it with an amino acid of approximately equivalent size, charge and/or polarity.

As used herein, the term “biocompatible” when used in relation to a material, such a protein or peptide, or variant thereof, means the material not substantially harmful or toxic to living tissue.

The terms “native” and “wild type” are used interchangeably herein and refer to a sequence that is normally found in nature.

The terms “polypeptide”, “peptide” and “protein” are used interchangeably herein and refer in their broadest sense to a molecule of two or more amino acid residues, or amino acid analogs. The amino acid residues may be linked by peptide bonds, or alternatively by other bonds, e.g., ester, ether etc., but in most cases will be linked by peptide bonds. The terms “amino acid” or “amino acid residue” are used herein to encompass both natural and unnatural or synthetic amino acids, including both the D- or L-forms, and amino acid analogs. An “amino acid analog” is to be understood as a non-naturally occurring amino acid differing from its corresponding naturally occurring amino acid at one or more atoms. For example, an amino acid analog of cysteine may be homocysteine.

The term “self-assembly” typically refers to a process in which a system of separate, pre-existing components, under specific conditions, adopts a more ordered and/or functional structure through interactions between the components themselves without external direction. In the context of the present invention, self-assembly typically refers to the spontaneous arrangement of one more materials (e.g., peptides, proteins, or functional variants thereof) into a hydrogel.

As used herein, the term “scaffold material” refers to a material capable of forming a biocompatible hydrogel scaffold, preferably a nanofibrillar hydrogel scaffold. Such materials may include, but are not limited to, peptides and functional variants thereof, natural or synthetic polymers, or combinations thereof. It is to be understood that combinations of scaffold materials may be used to provide composite hydrogels.

As used herein, the term “variant” typically refers to a structural protein comprising an amino acid sequence having at least 80% sequence identity to the amino acid sequence of the reference (e.g., native) protein. Reference to “at least 80% sequence identity” includes 80%, 85%, 86%, 87%, 88%, 89%, 90%, 91%, 92%, 93%, 94%, 95%, 96%, 97%, 98%, or 99% sequence identity to a reference sequence, for example, after optimal alignment or best fit analysis. In an embodiment, the variant comprises an amino acid sequence that has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or preferably at least 99% to the corresponding reference sequence, after optimal alignment or best fit analysis.

As used herein, the term “functional variant” typically refers to a polypeptide that has a different amino acid sequence to the reference polypeptide to which it is compared, including a natural (i.e., native) sequence or a synthetic variant thereof, yet retains at least some of the function ascribed to the reference molecule. Suitable methods of determining whether a variant retains the function of the native sequence will be familiar to persons skilled in the art. A functional variant may include a polypeptide sequence that differs from the reference sequence (e.g., a native sequence) by one or more (e.g., at least 1, at least 2, at least 3, at least 4, at least 5, etc.) amino acid substitutions, deletions, insertions or inversions, wherein the difference does not, or does not completely, abolish the functional ability of the variant.

As used herein, the terms “identity”, “sequence identity”, “homology”, “sequence homology” and the like mean that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. This may be referred to as conservative substitution. In an embodiment, the amino acid sequences may be modified by way of conservative substitution of any of the amino acid residues contained therein, such that the modification has no effect on the function of the modified polypeptide or protein when compared to the unmodified polypeptide or protein.

In some embodiments, sequence identity with respect to a peptide sequence relates to the percentage of amino acid residues in the candidate sequence that are identical with the residues of the corresponding peptide sequence after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology. Methods and computer programs for performing an alignment of two or more amino acid sequences and determining their sequence identity or homology are well known to persons skilled in the art. For example, the percentage of identity or similarity of two amino acid sequences can be readily calculated using algorithms, for example, BLAST, FASTA, or the Smith-Waterman algorithm.

Techniques for determining an amino acid sequence “similarity” are well known to persons skilled in the art. In general, “similarity” means an exact amino acid to amino acid comparison of two or more peptide sequences or at the appropriate place, where amino acids are identical or possess similar chemical and/or physical properties such as charge or hydrophobicity. A so-termed “percent similarity” then can be determined between the compared peptide sequences. In general, “identity” refers to an exact amino acid to amino acid correspondence of two peptide sequences.

Two or more peptide or protein sequences can also be compared by determining their “percent identity”. The percent identity of two sequences may be described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986). Suitable programs for calculating the percent identity or similarity between sequences are generally known in the art.

Unless otherwise specified, the indefinite articles “a”, “an” and “the” as used herein, include plural aspects. Thus, for example, reference to “an agent” includes a single agent, as well as two or more agents; reference to a “composition” or “formulation” includes a single composition or formulation, as well as two or more compositions or formulations; and so forth.

As used herein, the term “about” typically means±10% of the recited value.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “consisting of” means “consisting only of”, that is, including and limited to the integer or step or group of integers or steps, and excluding any other integer or step or group of integers or steps.

The term “consisting essentially of” means the inclusion of the stated integer or step or group of integers or steps, but other integer or step or group of integers or steps that do not materially alter or contribute to the working of the invention may also be included.

The disclosure of every patent, patent application, and publication cited herein is hereby incorporated herein by reference in its entirety.

The reference to any prior art in this specification is not, and should not be taken as, an acknowledgment or any form of suggestion that prior art forms part of the common general knowledge.

### DETAILED DESCRIPTION

The present invention relates to biocompatible hydrogels suitable for delivering a cell into a subject. Hydrogels according to the present invention may bind and release oxygen and/or scavenge reactive oxygen species (ROS) via the non-covalent binding of an oxygen carrier, such as myoglobin, to the hydrogel scaffold. Such hydrogels may be particularly advantageous in tissue regeneration, as they can be easily administered to a site of therapeutic need, such as a cell transplant site, where they can rapidly and effectively fill voids to ensure good tissue contact. In particular, certain hydrogels according to the present invention may modulate cell fate specification within progenitor cell grafts, resulting in a significant increase in neuronal differentiation, which may be useful, for example, in the treatment or prevention of neurodegeneration, such as that associated with Parkinson's disease.

### Hydrogels

Thus, the present invention broadly provides biocompatible hydrogels for delivering a cell into a subject, the hydrogel comprising a scaffold and an oxygen carrier non-covalently bound to the scaffold. Advantageously, hydrogels of the present invention may provide sustained presentation of oxygen to the cell or a population of cells. As used herein, sustained presentation of oxygen refers to the release of oxygen over a period of time. Preferably, hydrogels according to the present invention provide release of oxygen, preferably steady release, for at least about 5, 10, 15, 20, 25 or 28 days, or more.

Hydrogels according to the present invention may comprise a scaffold prepared using any suitable scaffold material. For example, suitable scaffold materials may comprise one or more peptides, naturally occurring polymers (including macromolecules such as polysaccharides, proteins and polynucleotides), synthetic polymers (such as polyamides and polyethylene glycol), or combinations thereof. In an embodiment, the hydrogel scaffold is covalently bound to a surface (e.g., a polymer surface, metal surface, ceramic surface, or the like). In embodiments in which the scaffold material is a peptide, it may comprise any functional amino acid sequence of two or more amino acids (e.g., 2, 3, 4, 5, 6 or more amino acids). Preferably, the scaffold material mimics cellular microenvironments to enable the survival, movement, differentiation and/or integration of transplanted cells. Thus, in a preferred embodiment, the hydrogel scaffold is a peptide-based scaffold. The peptide-based scaffold may comprise or consist of one or more peptides, preferably native peptides or functional variants thereof. Such peptides and/or functional variants thereof may be capable of self-assembly in the presence of water to form a self-assembled peptide (SAP) hydrogel having a nanofibrillar scaffold (also referred to as a “nanoscaffold”).

In the context of the present invention, such functional variants may consist of any peptide that has a different amino acid sequence to the native peptide but retains the ability to form of a hydrogel scaffold and non-covalently bind an oxygen carrier. Thus, the peptide functional variants may comprise one or more (e.g., 1, 2, 3, 4, 5 or more) amino acid substitutions, deletions, insertions and/or inversion relative to the native peptide, provided that none of those substitutions, deletions, insertions and/or inversions destroy the ability of the variant to form a hydrogel scaffold and non-covalently bind an oxygen carrier. In an embodiment, the functional peptide variant may differ from the native sequence by one or more conservative amino acid substitutions. In some embodiments, a functional peptide variant may include one or more amino acid substitutions and/or other modifications in order to increase the stability and/or to increase the solubility of the peptide relative to its native form. Suitable modifications will be apparent to those skilled in the art. Functional peptide variants may comprise an amino acid sequence having at least 80% sequence identity to the corresponding native peptide. For example, the amino acid sequence of the functional peptide variant may have at least 80%, 85%, 90%, 91%, 92%, 93%, 94%, 95%, 96%, 97%, 98%, or at least 99% sequence identity to the native peptide, after optimal alignment or best fit analysis. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference may also be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

In a preferred embodiment, the peptide-based scaffolds described herein comprise or consists of an extracellular matrix (ECM) protein or a functional variant thereof, or any combination thereof. The ECM is a complex network of fibrillar proteins and glycosaminoglycans, which provides cells with information on their environment. Thus, ECMs (and functional variants thereof) may be particularly useful in the hydrogels of the present invention because they are naturally occurring in the body and may therefore be degraded by enzymes naturally found in the body and then absorbed. Any naturally occurring ECMs may be suitable for use in the hydrogels of the present invention, including but not limited to laminin, collagen, fibrin, fibronectin, gelatin, elastin, hyaluronan, proteoglycans, polysaccharides, enzymes and integrins, or functional variants thereof, or any combination thereof. Such ECM proteins may self-assemble to form a hydrogel having a nanofibrillar scaffold. One or more ECM proteins (or functional variants thereof) may be used in combination to form a composite hydrogel, e.g., comprising one, two or more ECM proteins, that co-assemble or are mixed post-assembled to form a nanofibrillar scaffold. For example, two or more ECM proteins or functional variants thereof may be selected for the hydrogel scaffolds disclosed herein to more effectively mimic the native ECM, increase bioactivity and/or improve cellular response in vitro or in vivo, and may be prepared, for example, according to the methods of Horgan et al., 2016. Thus, the choice of ECM proteins(s) (or functional variants thereof) as a scaffold material for the hydrogels of the present invention may depend on the intended use of the hydrogel. For example, laminin is the brain's major ECM protein and thus a peptide-based comprising laminin or functional variant thereof may be particularly suitable for use when the hydrogel is intended for administration to the brain. Suitable peptides, including ECM proteins or functional variant thereof, for use in the present invention will apparent to those skilled in the art. In an embodiment, the ECM protein is laminin or a functional variant thereof.

Functional ECM variants suitable for use in the present invention may comprise or consist of an epitope region (binding domain) of the ECM. For example, it has previously been demonstrated that a peptide-based hydrogel may be prepared comprising the binding domain of laminin (i.e., IKVAV) in high density on the surface of a nanofibrillar molecular hydrogel substructure (Horgan et al., 2016; Maclean et al., 2018; Nisbet et al., 2018). Such domains of many proteins, such as ECM proteins, and methods for obtaining them are well-known in the art, or can be readily identified by those skilled in the art using standard techniques, such as X-ray co-crystallography or site-directed mutagenesis. The choice of ECM protein variant may depend on the type tissue to be treated and the predominant ECM proteins found in that tissue. For example, a functional (e.g., binding) domain of laminin (IKVAV or YIGSR) and/or fibronectin (RGD) may be selected for use with brain tissue, whereas the PHSRN sequence of fibronectin may be more suitable for use in bone. A person skilled in the art will be able to select suitable ECMs or binding domains thereof, depending on the tissue to be treated. The binding domain sequence may comprise one or more additional amino acids at one or both terminal ends.

Functional ECM variants, including functional ECM variants, suitable for use in the hydrogels of the present invention may be chemically functionalized peptide variants. Chemical functionalization may be used improve or impart one or more desirable properties to the hydrogel. For example, functional groups that facilitate self-assembly of the hydrogel may be incorporated into the peptide. By way of non-limiting example, a fluorenylmethoxycarbonyl (Fmoc) group may in incorporated into a peptide to enable pi-pi stacking, whereupon the peptide components align to form a network of beta sheets. Thus, Fmoc functionalization may facilitate self-assembly of a peptide-based hydrogel scaffold as described herein. Thus, in a preferred embodiment, the functional ECM variant is an Fmoc-functionalised epitope region of an ECM, e.g., Fmoc-DDIKVAV, Fmoc-DDIKVAVD or Fmoc-FRGDF, or any combination thereof. Functional ECM variants having a net charge, such as Fmoc-DDIKVAVD (net negative charge), may be particularly useful for binding an oxygen carrier to the hydrogel scaffold using electrostatic forces. Such functional ECM variants may be prepared, for example, using solid phase peptide synthesis (see, e.g., Horgan et al., 2016). Other suitable functional groups and methods for functionalizing peptides will be apparent to those skilled in the art. In an embodiment, the ECM protein is a functional variant of laminin. In an embodiment, the functional variant of laminin is IKVAV or YIGSR. In an embodiment, the functional variant of laminin is IKVAV. In an embodiment, the functional variant of laminin is YIGSR.

It is to be understood that the term “oxygen carrier” as used herein refers to compounds that is capable of both carrying and releasing oxygen. The oxygen carrier may be any suitable oxygen carrier, preferably an oxygen carrier protein or functional variant thereof. Such proteins will be apparent to those skilled in the art and may include, but are not limited to, myoglobin, haemoglobin, neuroglobin and cytoglobin, or a functional variant thereof, or any combination thereof. In a preferred embodiment, the oxygen carrier protein is myoglobin (Mb) or a functional variant thereof. Mb natively facilitates oxygen transport along partial pressure of oxygen (PO2) gradients and serves as an oxygen reservoir, binding oxygen via the prosthetic heme group in high oxygen concentrations (oxymyoglobin), and releasing oxygen in hypoxic conditions (deoxymyoglobin), such as those experienced during periods of increased metabolic activity. Moreover, during hypoxic or anoxic conditions, Mb scavenges potentially cytotoxic reactive oxygen species (ROS), such as peroxide and nitric oxide, which are by-products of oxygen metabolism. Such homeostatic properties make Mb an ideal O₂ vector/reservoir for stem cell grafts.

Previous studies have shown that Mb can be imbibed or immobilised within sol/gel films (Burke et al., 2017; Castro-Forero et al., 2008). In contrast to such covalent protein immobilization strategies that require chemical modifications to the hydrogel, the present inventors have surprisingly found that oxygen carriers, such as Mb, can be non-covalently (e.g., electrostatically) bound to a hydrogel scaffold as described herein without adversely affecting the biophysical properties and/or self-assembly of the hydrogel. Without being bound by theory or mode of application, it is understood that hydrogels according to the present invention undergo two-component assembly, whereby the scaffold material(s) assemble to form a hydrogel and the oxygen carrier (e.g., myoglobin) molecules associate with the surface of, but do not disrupt the structure of, the hydrogel. Such non-covalent binding of the oxygen carrier in the hydrogel may improve efficacy of delivery and allow for sustained presentation of oxygen to a cell or population of cells.

Thus, in accordance with the present invention, the oxygen carrier is non-covalently bound to the hydrogel scaffold. Non-covalent binding may include electrostatic binding, ionic binding, hydrophobic binding, hydrogen bonding, among others. In a preferred embodiment, the oxygen carrier is electrostatically bound to the scaffold. The non-covalently binding of an oxygen carrier to the hydrogel scaffold may, for example, result in more extensive innervation within the host tissue from the grafted cells, which is essential for neuronal replacement strategies to ensure functional synaptic connectivity. Accordingly, hydrogels of the present invention may provide greater functional integration of stem cell-derived grafts for the treatment of neural injuries and diseases affecting the central and peripheral nervous systems.

In the context of the present invention, functional oxygen carrier protein variants may, for example include oxygen carrier proteins having a different amino acid sequence to the native oxygen carrier protein but retains the ability to carry and release oxygen, and to non-covalently bind to a hydrogel scaffold as disclosed herein. Thus, the oxygen carrier protein variants may comprise one or more (e.g., 1, 2, 3, 4, 5 or more) amino acid substitutions, deletions, insertions and/or inversion relative to the native protein, provided that none of those substitutions, deletions, insertions and/or inversions destroy the ability of the variant to carry and release oxygen, and to non-covalently bind to a hydrogel scaffold as disclosed herein. In the context of the present invention, such functional variants may consist of any oxygen carrier protein that has a different amino acid sequence to the native protein but retains the ability to carry. In an embodiment, the functional oxygen carrier protein variant may differ from the native sequence by one or more conservative amino acid substitutions. In some embodiments, a functional oxygen carrier protein variant may include one or more amino acid substitutions and/or other modifications in order to increase the stability and/or to increase the solubility of the protein relative to its native form. Suitable modifications will be apparent to those skilled in the art. Functional oxygen carrier protein variants may comprise an amino acid sequence having at least 80% sequence identity to the corresponding native protein. For example, the amino acid sequence of the functional oxygen carrier protein variant may have at least 80%, 85%, 90%, 91%, 92%, 93%, 94%, 95%, 96%, 97%, 98%, or at least 99% sequence identity to the native peptide, after optimal alignment or best fit analysis.

The present inventors have found that functional oxygen carrier protein variants may be used to alter the oxygen release kinetics of a hydrogel of the present invention. For example, the present inventors have unexpectedly found that mutation of one or more amino acids of wild-type sperm whale myoglobin (Physeter microcephalus) can alter the oxygen binding affinity of the myoglobin, e.g., Leu29Phe mutation increased oxygen binding affinity, His64Leu mutation decreased binding affinity relative to the wild-type Mb. Thus, functional myoglobin variants suitable for use in the hydrogels of the present invention may include one or more amino acid mutations, including, but not limited to Leu29Phe and/or His64Leu mutations. Such mutations may be introduced, for example, by site directed mutagenesis or other suitable methods known in the art.

Peptides and proteins or functional variants thereof as described herein, may be synthetically produced by chemical synthesis methods that are well known in the art, either as an isolated peptide sequence or as a part of another peptide or polypeptide. Alternatively, the peptides/proteins, or functional variants thereof as described herein may be produced in a microorganism that produces the relevant protein sequence, which can then be isolated and, if desired, further purified. The protein sequences can be produced in microorganisms such as bacteria, yeast or fungi, in eukaryote cells such as a mammalian or an insect cell, or in a recombinant virus vector such as adenovirus, poxvirus, herpesvirus, Simliki forest virus, baculovirus, bacteriophage, sindbis virus or sendai virus. Suitable bacteria for producing the peptide or protein sequences will be familiar to persons skilled in the art, illustrative examples of which include E. coli, B. subtilis or any other bacterium that is capable of expressing the peptide sequences. Illustrative examples of suitable yeast types for expressing the peptide or protein sequences include Saccharomyces cerevisiae, Schizosaccharomyces pombe, Candida, Pichia pastoris or any other yeast capable of expressing peptides. Corresponding methods are well known in the art and are described elsewhere herein. Methods for isolating and purifying polypeptides are well known in the art and include, for example, gel filtration, affinity chromatography and ion exchange chromatography.

Peptides or proteins, and functional variants thereof, may also be produced using recombinant techniques. The production and expression of recombinant (fusion) proteins is well known in the art and can be carried out using conventional procedures, such as those disclosed in Sambrook et al., Molecular Cloning: A Laboratory Manual (4th Ed. 2012), Cold Spring Harbor Press. For example, expression of a fusion protein may be achieved by culturing recombinant host cells containing the nucleic acid encoding the fusion protein under appropriate conditions. Following production by expression, the fusion protein may be isolated and/or purified using any suitable technique, such as ion exchange chromatography, affinity chromatography and gel filtration, among others.

Hydrogels according to the present invention may further comprise a cell or cells. The type of cell(s) may depend on the site of transplantation and/or the disease to be treated and may include mammalian cells, stem, precursor and progenitor cells isolated from the adult or embryonic brain, neural subventricular zone, hippocampal subgranular zone, spinal cord, skin, blood, mesenchyme, umbilical cord, skin-derived precursors or adult retinal ciliary epithelium and their undifferentiated and differentiated progeny; embryonic stem cells and their undifferentiated and differentiated progeny; epiblast stem cells and their undifferentiated and differentiated progeny; primitive and definitive neural stem cells and their undifferentiated and differentiated progeny; induced pluripotent stem cells and their undifferentiated and differentiated progeny; mesenchymal stem cells and their undifferentiated and differentiated progeny; bone-marrow derived stem cells and their undifferentiated and differentiated progeny; hematopoietic stem cells and their undifferentiated and differentiated progeny; umbilical cord derived stem/progenitor cells and their undifferentiated and differentiated progeny; neural precursor cells of the forebrain, midbrain, hindbrain, spinal cord, neural crest, and retinal precursors isolated from developing tissue and their undifferentiated and differentiated progeny. A skilled person will be readily able to identify the appropriate cell or cells for use with the hydrogels of the present invention depending on the circumstances. For example, cell transplantation for Parkinson's disease involves dopaminergic neurons, generally sourced from ventral midbrain fetal tissue. In an embodiment, the cell is a stem cell. In an embodiment, the stem cell is a neural precursor cell.

Hydrogels according to the present invention may further comprise one or more therapeutic agents. Non-limiting examples of suitable therapeutic agents may include anaesthetics (e.g., propofol, etomidate, methohexital, thiopentone/thiopental, midazolam, ketamine); analgesics (e.g., acetaminophen, ibuprofen, fluriprofen, ketoprofen, voltaren, phenacetin, salicylamide), anti-inflammatories (e.g., naproxen, indomethacin), antihistamines (e.g., chlorpheniramine maleate, phenindamine tartrate, pyrilamine maleate, doxylamine succinate, henyltoloxamine citrate, diphenhydramine hydrochloride, promethazine, brompheniramine maleate, dexbrompheniramine maleate, clemastine fumarate, triprolidine); antibiotics (e.g., penicillin, cefepime, moxifloxacin, vancomycin, metronidazole, linezolid); central nervous system drugs (e.g., thioridazine, diazepam, meclizine, ergoloid mesylates, chlorpromazine, carbidopa, levodopa); metal salts (e.g., potassium chloride, lithium carbonate); minerals (e.g., iron, chromium, molybdenum, potassium); immunomodulators; immunosuppressants (e.g., minocycline, cyclosporine A); peptide and glycoprotein hormones and analogues thereof (e.g., human chorionic gonadotrophin (HCG), corticotrophin, human growth hormone (HGH-Somatotrophin) and erythropoietin (EPO); steroids and hormones (e.g., ACTH, anabolics, androgen and estrogen combinations, androgens, corticoids and analgesics, estrogens, glucocorticoid, gonadotropin, gonadotropin releasing, hypocalcemic, menotropins, parathyroid, progesterone, progestogen, progestogen and estrogen combinations, somatostatin-like compounds, urofollitropin, vasopressin, methyl prednisolone, and GM1 ganglioside); small molecules (e.g., taurine, retinoic acid, sodium butyrate, cAMP, dbcAMP); vitamins; growth factors, proliferative factors and morphogens (e.g., VEGF, bFGF, PR11, PR39, SDF-1, IGF); neurotrophic factors (e.g., GDNF, BDNF); stem cell homing factors (e.g., GSCF) peptides, peptide mimetics and other protein preparations; anti-angiogenics (e.g., ranibizumab, bevacizumab, pegaptanib), amino acids, proteins, among others. Other suitable components with be apparent to those skilled in the art any my selected for their in vitro or in vivo properties depending on the intended use of the hydrogel. For example, growth factors, such as vascular endothelial growth factor (VEGF) or basic fibroblast growth factor (bFGF), may be incorporated into the hydrogel to elicit new microvessel growth in vivo. Such therapeutic agents are preferably homogenously and non-covalently distributed throughout the hydrogel as micron-sized particles.

The present invention also provides methods for preparing hydrogels as disclosed herein. The hydrogels may be formed by combining a scaffold material, an oxygen carrier and water using any suitable method, including but not limited to post-addition (i.e., via shear entrapment of the oxygen carrier into the liquid component of a preformed hydrogel scaffold), pre-addition (i.e., via incorporation of the oxygen carrier as a starting component of the assembly) or per-addition (i.e., via introduction of the oxygen carrier during later stages of assembly to interact with the surface of the hydrogel scaffold).

For example, the present invention provides a method for preparing a biocompatible hydrogel as disclosed herein, comprising:
a) combining a scaffold material and water to form a hydrogel scaffold;
b) combining the hydrogel scaffold with an oxygen carrier to form a homogenous mixture;
c) gelation of the homogenous mixture to form a biocompatible hydrogel in which the oxygen carrier is non-covalently bound to the hydrogel scaffold.

Preferably, step (a) comprises dissolving the scaffold material, preferably a peptide or functional variant thereof, in deionised water, more preferably sterile deionised water suitable for injection. In an embodiment, the peptide may be dissolved in the minimum amount of water required for complete dissolution, optionally in the presence of base (e.g., NaOH). If necessary, the resulting solution may be adjusted to physiological pH (e.g., using a suitable acid, such as HCl). In an embodiment, water or an aqueous solution (e.g., PBS buffer, HBBS) may be added to the solution form a hydrogel at a desired concentration, for example, about 5-30 mg mL-1, or about 10-20 mg mL-1 (e.g., about 15 mg mL-1) of the scaffold material and the mixture homogenized, e.g., by stirring or vortexing. Preferably, gelation of the homogenous mixture occurs spontaneously, e.g., by self-assembly, upon resting of the mixture at ambient temperature. Following formation of the peptide-based hydrogel, step (b) comprises adding an oxygen carrier as described herein, preferably an oxygen carrier protein or a functional variant thereof (or combination thereof). The oxygen carrier may be solubilised in aqueous solution (e.g., using water or PBS buffer) before being added to the hydrogel scaffold at a suitable concentration, e.g., about 0.5-5 mg mL-1, or about 0.5-2 mg mL-1 (e.g., about 1 mg mL-1). Preferably, the resulting mixture is mixed until substantially homogenised before regelation (e.g., by self-assembly) of the homogenous mixture upon resting at ambient temperature.

The present invention also provides a method for preparing a biocompatible hydrogel as disclosed herein, comprising:
a) combining a peptide or a functional variant thereof, an oxygen carrier, and water to form a homogenous mixture;
b) gelation of the homogenous mixture to form a hydrogel in which the oxygen carrier is non-covalently bound to a peptide-based scaffold.

Such reagents, conditions, concentration, etc., as described above for two component assembly of the hydrogels disclosed herein may also be suitable for use in the pre-addition method.

The methods for preparing a biocompatible hydrogel as disclosed herein may further comprise incorporating cells and/or one or more therapeutic agents into the hydrogel. For example, cells and/or therapeutic agents may be added during any step of the methods disclosed herein, preferably in a liquid state of the hydrogel. If necessary, a hydrogel (or intermediate hydrogel scaffold) may be liquefied, e.g., by vigorous stirring or vortexing, together with the cells and/or therapeutic agent(s) until a homogenous mixture is achieved, followed by regelation, whereupon the cells and/or therapeutic agent(s) are incorporated into the hydrogel structure by shear entrapment.

### Therapeutic Use

The biocompatible hydrogels of the present invention may be suitable for delivering a cell into a subject, where they may provide sustained presentation of oxygen to the cell. The hydrogels disclosed herein may also be suitable for in vitro use. In particular, it is envisaged that hydrogels of the present invention may be administered to the site of a cell transplant (e.g., a stem cell transplant) in a subject. Thus, the present invention provides a method for transplanting a cell into a subject, the method comprising administering the biocompatible hydrogel as described herein to the tissue. The present invention also provides use of a biocompatible hydrogel as described herein in the manufacture of a medicament for transplanting a cell into a subject.

The subject may be any animal in need of cell transplant and encompasses human and non-human subjects, including, but not limited to, mammals, birds and fish, and suitably encompasses domestic, farm, zoo and wild animals, such as, for example, cows, pigs, horses, goats, sheep or other hoofed animals, dogs, cats, chickens, ducks, non-human primates, guinea pigs, rabbits, ferrets, rats, hamsters and mice. Preferably, the subject is a mammal, more preferably a human.

The terms “administer”, “administering” or “administration” in reference to a hydrogel disclosed herein means introducing the hydrogel into tissue of the subject in need of treatment. In general, the hydrogel may be administered in a manner compatible with the route of administration and physical characteristics of the recipient (including health status) and in such a way that it elicits the desired effect(s). When the hydrogel is provided in combination with one or more therapeutic agents, “administration” and its variants are each understood to include concurrent and/or sequential introduction of the hydrogel and the active agent(s). The hydrogels disclosed herein may be administered in a single dose or a series of doses.

The subject may be in need of a cell transplant for the treatment of a disease a disorder such as a neurodegenerative disease, cancer, stroke, genetic disorder, liver disorder, development disorder, degenerative disorder, familial or traumatic disorders of the nervous system, vascular disorder, skin disease, skin disorder, auto immune disorder, eye disorder, kidney disorder, cardiac disorder, musculoskeletal disorder, reproductive disorder, fertility disorder, or blood disorder. Suitable cell types for the treatment of a particular disease or disorder will be apparent to those skilled in the art. Preferably, the hydrogel comprises cells to be transplanted, although it also envisaged that the hydrogel may be delivered to the transplant site concomitantly with the cells to be transplanted (e.g., before, at the same time as, or after transplantation of the cells).

The hydrogels disclosed herein are to be administered to a subject in need thereof in an effective amount. In the context of the present invention, an “effective amount” relates to an amount of hydrogel that, when administered to tissue of a subject, provides the desired therapeutic activity. For example, an effective amount of a hydrogel may be an amount sufficient to improve the survival, differentiation and/or integration of cells in the tissue of the subject. Suitable effective amounts may depend on the age, gender, weight and general health of the patient and can be determined by the attending physician. Advantageously, hydrogels as described herein may provide sustained presentation of oxygen to cells in tissue of a subject for a suitable duration (e.g., at least about 28 days or more) to improve survival, differentiation and/or integration of the cells (e.g., stem cells).

The biocompatible hydrogels as described herein may be suitable for administration to any site of a cell or cell population in the tissue of a subject requiring sustained oxygen presentation, including but not limited to cell transplants in the brain, spinal cord, heart, lung, kidney, liver, eye, ear, bone, cartilage, tendon, skin, or any other organ or tissue in need of a cell transplant. The hydrogels described herein may be administered to a subject in need thereof by any suitable route of administration, which may depend on the site of the cell(s).

Biocompatible hydrogels according to the present invention may be particularly useful for sustained presentation of oxygen to stem cells transplanted for the treatment or prevention of an injury, disease or infection of the central nervous system (CNS), including neurodegenerative diseases such as Parkinson's disease and Alzheimer's disease, Bell's palsy, cerebral palsy, epilepsy, motor neuron disease (MND), multiple sclerosis (MS), neurofibromatosis, dementia, among others, as well as brain or spinal cord injuries, including stroke, cancer and iatrogenic injury. Biocompatible hydrogels according to the present invention may also be useful for providing sustained presentation of oxygen to stem cells transplanted for the treatment or prevention of an injury, disease or infection of the peripheral nervous system (PNS), including Sjogren's syndrome, lupus, rheumatoid arthritis, Guillain-Barre syndrome, chronic inflammatory demyelinating polyneuropathy and vasculitis. Such injuries may be primary or secondary injuries, which may result in rapid necrotic death of cells and degradation of the local ECM, which in turn can lead to collapse or distortion of surrounding tissue that inhibit regeneration. Typically, cell transplantation for the treatment of a disease or disorder of the CNS or PNS requires injection of the cells into the tissue of a subject in need of treatment, particularly tissue comprising a lesion of the CNS or PNS. For example, stem cell transplantation for the treatment of Parkinson's disease typically involves injection of the cells onto to the striatum. Thus, the hydrogels described herein are preferably injectable hydrogels.

The terms “treat”, “treating” or “treatment” with regard to a viral infection refers to alleviating or abrogating the cause and/or the effects of the viral infection. As used herein, the terms “treat”, “treatment” and “treating” refer to the reduction or amelioration of the progression, severity and/or duration of the viral infection, or the amelioration of one or more symptoms (e.g., one or more discernible symptoms) of the viral infection (i.e., “managing” without “curing” the condition), resulting from the administration of one or more therapies (e.g., one or more therapeutic agents such as a compound or composition as disclosed herein). In specific embodiments, the terms “treat”, “treatment” and “treating” refer to the amelioration of at least one measurable physical parameter of a viral infection described herein. In other embodiments the terms “treat”, “treatment” and “treating” refer to the inhibition of the progression of a viral infection described herein, either physically by, e.g., stabilization of a discernible symptom or physiologically by, e.g., stabilization of a physical parameter, or both.

The terms “preventing” and “prophylaxis” as used herein refer to administering a medicament in order to avert or forestall the appearance of one or more symptoms of a condition. The person of ordinary skill in the medical art recognizes that the term “prevent” is not an absolute term. In the medical art, it is understood to refer to the prophylactic administration of a drug to substantially diminish the likelihood or seriousness of a condition, or symptom of the condition and this is the sense intended in this disclosure. As used in a standard text in the field, the Physician's Desk Reference, the terms “prevent”, “preventing” and “prevention” with regard to a condition refer to averting the cause, effects, symptoms or progression of a condition prior to the condition fully manifesting itself.

Where a hydrogel according to the present invention comprises cells for transplantation, the number of cells is preferably a therapeutically or prophylactically effective amount. The term “therapeutically effective amount” as used herein means an amount of cells sufficient to treat or alleviate the symptoms associated with the disease or disorder for which the cell transplant is indicated. The term “prophylactically effective amount” refers to an amount effective in preventing or substantially lessening the chances of acquiring a disease or disorder or in reducing the severity of the disease or disorder before it is acquired or reducing the severity of one or more of its symptoms before the symptoms develop. Roughly, prophylactic measures are divided between primary prophylaxis (to prevent the development of a disease or symptom) and secondary prophylaxis (whereby the disease or symptom has already developed and the patient is protected against worsening of this process). A skilled person will be able to determine a therapeutically or prophylactically effective amount depending on the disease or disorder for which the cell transplant is indicated and is typically between about 10,000 cells and about 200 million cells. Such cells are preferably homogenously distributed throughout the hydrogel.

Hydrogels according to the present invention may be administered in combination with one or more therapeutic agents. Such therapeutic agents may be incorporated into the hydrogel as described elsewhere herein, and/or administered as a combination therapy with a hydrogels according to the present invention (administered sequentially or in combination). Such combination therapy may be particularly useful, for example, where an additive or synergistic therapeutic effect is desired. The phrase “combination therapy” as used herein, is to be understood to refer to administration of an effective amount, using a first amount of, for example, a hydrogel as described herein, and a second amount of a therapeutic agent. The hydrogels disclosed herein may be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

In one or more embodiments where the hydrogel is administered with a therapeutic agent, the active agent may be any therapeutic agent that provides a desired treatment outcome. In particular, the additional therapeutic agent may be selected from known therapeutic agents for the treatment or prevention of the disease or disorder from which a cell transplant is indicated, including one or more symptoms thereof. By way of example only, the therapeutic agent may be a known therapeutic agent for the treatment of Parkinson's disease, such as a levodopa preparation (e.g., levodopa/benserazide, levodopa/carbidopa, levodopa/benserazide, levodopa/carbidopa, levodopa/benserazide), a dopamine agonist (e.g., bromocriptine, cabergoline, pergolide, pramipexole, ropinirole, apomorphine), a catechol-O-methyltransferase inhibitor (e.g., entacapone, tolcapone, a monoamine oxidase B inhibitor (e.g., selegiline), an NMDA antagonist (e.g., amantadine) or an anticholinergics (e.g., benzhexol, benztropine, biperiden, orphenedrine, procyclidine. Suitable therapeutic agents may be selected by those skilled in the art depending on the circumstances, including the particular disease or disorder from which a cell transplant is indicated. Where a hydrogel is administered in combination with a therapeutic agent, the active agent may be administered in any “effective amount” that provides the desired therapeutic activity, as described above. Suitable dosage amounts and dosing regimens of the additional therapeutic agent can be determined by the attending physician and may depend on the particular condition being treated, the severity of the condition as well as the general age, health and weight of the subject.

The biocompatible hydrogels or components thereof as disclosed herein may be contained in a kit. The kit may include, for example, the hydrogel together with an instrument for assisting with the administration of the composition to a patient, e.g., a syringe. Alternatively, the kit may comprise the hydrogel and cells and/or one or more therapeutic agents, each packaged or formulated individually, or packaged or formulated in combination. Thus, the hydrogels may be present in first container, and the kit can optionally include one or more cells and/or active agents in separate container(s). The container or containers may be placed within a package, and the package can optionally include administration or dosage instructions. In another embodiment, the kit may comprise the components of the hydrogel (e.g., peptide and oxygen carrier) in dried or lyophilised form and the kit can additionally contain a suitable solvent (i.e., water) for reconstitution of the lyophilised components to form a hydrogel. The kits may optionally comprise instructions describing a method of using the hydrogels in one or more of the methods described herein (e.g., for delivering a cell to a subject).

Those skilled in the art will be aware that the invention described herein is subject to variations and modifications other than those specifically described. It is to be understood that the invention described herein includes all such variations and modifications. The invention also includes all such steps, features, methods, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### General Procedures

### A. Transmission Electron Microscopy (TEM)

Negative-staining transmission electron microscopy (TEM) was performed using a HITACHI HA7100 TEM with a LaB6 cathode at 125 kV (tungsten filament). Formvar-coated copper grids were prepared with electron glow discharge at 15 mA for 30 seconds. The formvar-coated side of grids was loaded with hydrogel for 30 seconds, washed with DI H₂O (20 μL), treated with urea-formaldehyde (UF, 20 μL), and finally immersed into UF drop for 30 seconds. Between each step, excess solution was blotted off using filter paper. Then, the grids were allowed to dry overnight before imaging in the TEM.

### B. Fourier Transform Infrared Spectroscopy (FTIR)

Fourier transform infrared spectroscopy (FTIR) was performed using an Alpha Platinum Attenuated Total Reflectance (ATR) FTIR (Bruker Optics) to monitor interactions in Amide Iregion (1550 to 1750 cm-1). Approximately 20 μL of peptide hydrogel was placed on the single reflection diamond. Absorbance scans were obtained for each peptide, and a background buffer scan subtracted.

### C. Circular Dichroism (CD)

Circular dichroism (CD) was performed using a Chirascan CD Spectrometer (Applied Photophisics Limited) to determine the secondary structure of hydrogel. The hydrogel was diluted at 1:10 ratio of hydrogel and DI H₂O to reduce scattering effects. The diluted gel around 400 μL was added into the cuvette with a 10 mm path length. CD scans ranged from 180 nm to 320 nm with a step size of 0.5 bandwidths using a Chirascan CD spectrometer (Applied Photophysics Limited) and a baseline (DI H₂O) was subtracted. The resulting data were averaged and smoothed post-acquisition using Chirascan software.

### D. Small Angle X-Ray Scattering (SAXS)

SAXS was performed using SAXS/WAXS beamline at the Australian Synchrotron^{[32]}. Measurements were taken using camera length 900 mm, time exposure 1 second, energy 12 keV and 5% flux. Samples (SAPs groups, SAPs+ Myoglobin groups) were prepared as detailed above 1 day before measurement and stored in Eppendorf vials. PBS buffer was loaded into a 1 mm glass capillary for background measurements. Each hydrogel sample was loaded into six of the same capillaries for measurement. Capillaries were loaded into a custom mount which can hold and move the capillaries in two dimensions, with Kapton film windows. 1 s exposures were taken for each hydrogel-loaded capillary at 10 different positions evenly spread along the 3 mm capillary length. The average of these positions was used for one capillary. Each group of 6 capillaries for each sample was further averaged and the PBS background subtracted using ScatterBrain. There was strong scattering from the Kapton windows centred at ˜0.4 Å⁻¹, which marks the upper limit of the useable Q-range measured.

### E. Rheology

The rheological analysis was performed using a Kinexus Pro+ Rheometer (Malvern) and rSpace software. Approximately 0.2 mL of hydrogel was placed on a 20 mm roughened plate (with solvent trap, Lower Geometry: PLS55 C0177 SS, Upper Geometry: PU20 SR1351 SS). The gap size was 0.2 mm, and multiple frequency sweeps were performed for frequencies ranging from 0.1-100 Hz with a 0.1% oscillatory strain at a constant required temperature (37° C.). Each gel was allowed a minimum of 5 minutes to set before testing.

### F. Statistical Analysis

Results represent means±standard error of the mean (SEM). Data were analyzed using Graph Pad Prism 6.0 by one-way ANOVA with Turkey post-hoc statistic testing. Differences at P<0.05-0.01 were considered statistically significant.

### Example 1. Protein Expression, Purification, and Sequence Analysis

The sequences of Equus caballus (Horse Mb, SwissProt accession number: P68082) and Physeter macrocephalus (Sperm whale Mb, SwissProt accession number: P02185) were aligned with T-COFFEE server (Di Tommaso, et al. 2011) and the pI values were calculated using the Expasy ProtParam Tool (Bjellqvist et al., 1993).

Lyophilized horse myoglobin (Sigma) was reconstituted in phosphate buffered saline (pH 7.4) and further purified using size exclusion chromatography (HiLoad 26/600 Superdex 200; Cytiva). The protein eluted as a single dominant peak and SDS-PAGE analysis indicated that the protein was essentially pure. There was one small band at a molecular weight corresponding to dimer, which could have formed during heating of the samples for loading onto the gel; indeed, apo-horse myoglobin is known to form a dimer once the heme is lost (Nagao et al., 2012). Wild-type Physeter macrocephalus myoglobin cloned into pMB413a was a gift from Stephen Sligar (Addgene plasmid #20058; http://n2t.net/addgene: 20058; RRID: Addgene20058; Springer & Sligar, 1987). This plasmid was mutated using Gibson assembly mutagenesis (Gibson et al., 2009) to generate the Leu29Phe mutant using the following primers:

(SEQ ID NO: 1)
F: 5′-GTCGCTGGTCATGGTCAGGACATCTTCATTCGACTGTTCAAAT
CTCATCCGG-3′
(SEQ ID NO: 2)
R: 5′-CGGATGAGATTTGAACAGTCGAATGAAGATGTCCTGACCATGA
CCAGCGACG-3′

The His64Leu mutant was generated in an identical manner using the following primers:

(SEQ ID NO: 3)
F: 5′-GAAAGCTTCTGAAGATCTGAAAAAACTGGGTGTTACCGTGTTA
ACTGCCCTA-3′
(SEQ ID NO: 4)
R: 5′-TAGGGCAGTTAACACGGTAACACCCAGTTTTTTCAGATCTTCA
GAAGCTTTC-3′

Purified DNA from the Gibson assembly reaction was used to transform E. coli. The plasmid DNA from a single colony was extracted and the sequence of the mutants was confirmed by Sanger sequencing (Ramaciotti Centre for Genomics, Australia).

All proteins were expressed through transformation into BL21 (DE3) E. coli cells and grown for 20 h at 37° C. with shaking (200 rpm) in 1 L Lysogeny Broth (LB) medium supplemented with 100 mg ampicillin (Bertani, 1951). Cells were harvested through centrifugation (10,000×g). For lysis, the pellet was resuspended in 25 mM HEPES pH8, 1 mM EDTA, 0.5 mM DTT, lysed by sonication (Sonic Ruptor 400 Ultrasonic Homogenizer (Omni) at 50% power and 50% pulse length for 6 minutes whilst the cells were immersed in an ice bath) with one repeat after a 6 minute recovery at 4° C. Cell debris was removed by centrifugation (30,000×g) for 45 min at 4° C.) and filtration 0.45 μm-pore-size nitrocellulose membrane (Millipore) and the clarified supernatant was collected. Protein was purified through anion exchange chromatography (DEAE fractogel, Merck) equilibrated with 25 mM HEPES pH8, 1 mM EDTA, 0.5 mM DTT, and protein was eluted over a gradient in which the concentration of NaCl in the buffer was increased from 0 to 1.5 M NaCl. The purity of the eluted fractions were then analysed with SDS-PAGE The most pure myoglobin-containing fractions from anion exchange chromatography were then purified further and buffer was exchanged using size exclusion chromatography, which was performed in an identical manner as described for horse myoglobin (above). Pure monomeric fractions from size exclusion chromatography was concentrated to approximately 16 mg/mL using an Amicon Ultra-15 centrifugal filter 10 kDa (Millipore) and loaded into the column with a syringe. Protein concentration was determined using absorbance at 280 nm using an extinction coefficient of 15470 M⁻¹ cm⁻¹ for Physeter. macrocephalus myoglobin (UniProt acc. number P02185) and 13980 M⁻¹ cm⁻¹ for Equus caballus myoglobin (UniProt acc. number P68082).

### Example 2. Preparation of Self-Assembling Peptide Scaffolds

Fmoc-DDIKVAV was synthesised at 0.4 mmol scale by solid phase peptide synthesis using a rotating glass reactor vessel. All chemicals were purchased from Sigma Aldrich (Australia) with the amino acids being purchased from Pepmic (China). The Fmoc-DDIKVAV hydrogel were prepared at a final concentration of 15 mg mL-1 using a well-established pH switch. Approximately 10 mg of Fmoc-DDIKVAV was dissolved in 200 μL of deionised water with 100 μL 0.5 M sodium hydroxide (NaOH). Then 0.1 M of hydrochloric acid (HCl) was added dropwise with continuous vortexing until the solution reached physiological relevant pH (Oaktron pH 700 micro pH electrode, Thermo Scientific). 0.01 M Phosphate buffered saline (PBS) was added to final 15 mg mL-1 concentration of hydrogel. For in vitro use, Hank's buffered saline solution (HBSS) (Gibco) was used in place of the PBS.

Lyophilized horse skeletal muscle myoglobin (Equus caballus; Sigma) was reconstituted in a PBS buffer and purified further by size exclusion using a SEC200 26/600 column (GE Healthcare) and an AKTA FPLC (GE Healthcare). The protein eluted in a single peak and no additional protein bands were visible on SDS-PAGE. The purified protein was aliquoted into 500 μL samples before being freeze-dried and stored at −80° C. For the preparation of the myoglobin:hydrogel hybrid, 1.2 mg of myoglobin was solubilised in 90.09 μL PBS as the stock solution, then 50 μL of that sample was added into the hydrogel (prepared as described above) to a final concentration of 1 mg mL-1 myoglobin. The vial was vortexed (30 seconds) for homogenisation and rested (60 seconds) for re-gelation. The final hydrogel contained 15 mg mL⁻¹ peptide hydrogel and 1 mg mL⁻¹ myoglobin.

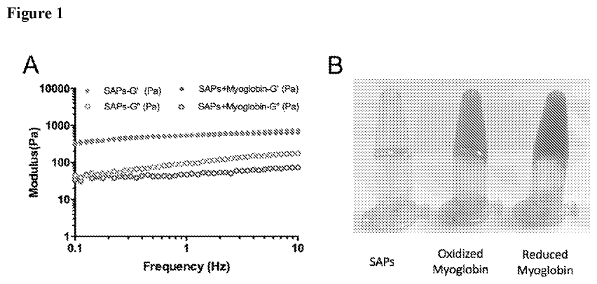

Characteristically red-coloured hydrogels were yielded, which were then optimised to be compliance matched to that of the rodent brain (G′=100 Pa, G″=100 Pa) (FIG. 1), as previously described in Rodriguez et al., 2013 and Rodriguez et al., 2018, the entire contents of which are incorporated herein by reference.

### Example 3. Spectral Characterisation of Myoglobin-Functionalised Hydrogels

Unfunctionalized Fmoc-DDIKVAV hydrogel (15 mg mL-1) and the same hydrogel functionalized with a final concentration of 1 mg mL-1 myoglobin that was reduced by reaction with excess sodium dithionite (Na₂S₂O₄; 17 mM) were prepared in a N₂-containing anaerobic hood (according to Example 1). Upon removal from the anaerobic hood, UV-Vis absorbance of the samples was monitored using a 96-well plate reading Epoch spectrophotometer (Biotek) over ten hours over a range of 350-700 nm. The same experiment was performed with free myoglobin in solution, in which everything in the system was identical, except for the absence of the hydrogel.

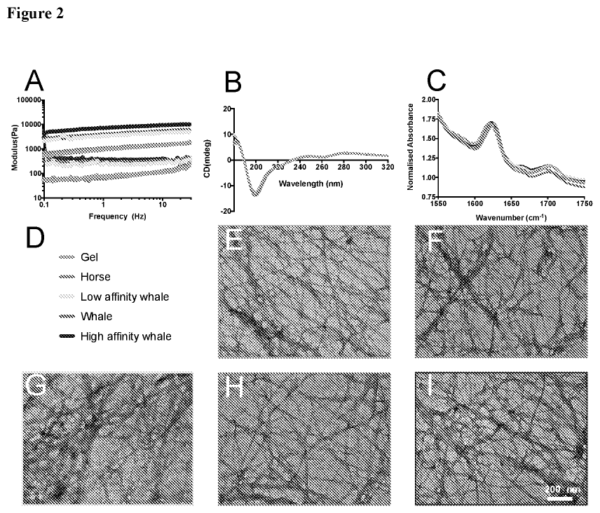

The addition of Mb resulted in minimal changes in the biophysical properties of the hydrogel as determined by transmission electron microscopy (TEM) according to General Procedure A, Fourier transform infrared spectroscopy (FTIR) according to General Procedure B, and circular dichroism (CD) according to General Procedure C (FIG. 2A-D). The results indicated that the system undergoes two component assembly, where myoglobin molecules associate with the surface of, but do not disrupt the structure of, the nanofibrils.

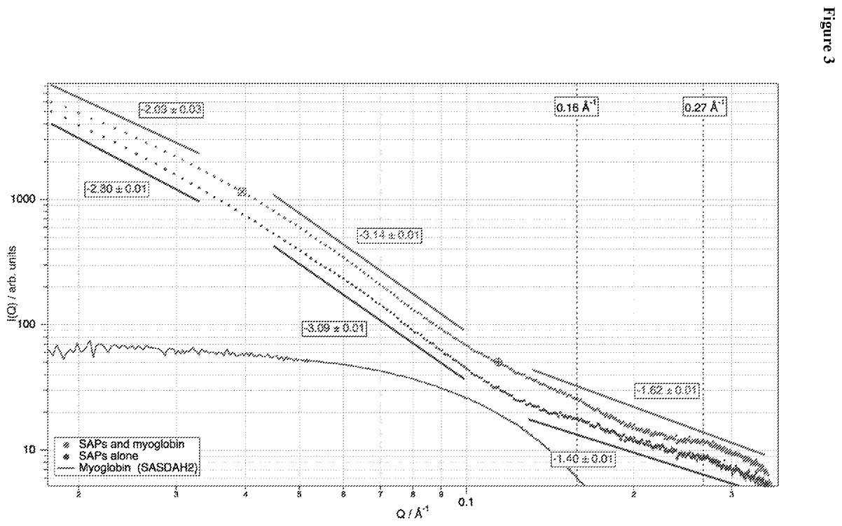

Those observations were confirmed by SAXS analysis of the hydrogel and myoglobin:hydrogel samples according to General Procedure D (FIG. 3). Both samples exhibited classic hydrogel SAXS scattering profiles, with a slope that is consistent with randomly oriented nanofibers in both samples and with minimal differences between the samples. Some weak scattering features at ˜0.16 Å-1 and 0.27 Å-1 (marked with dashed lines; real space sizes 39 Å and 23 Å respectively) are present in both samples and likely reflect cluster sizes in the hydrogels. The presence of those features in both the hydrogel and myoglobin:hydrogel samples indicates that the base structure of the self-assembled peptide (SAP) hydrogel is unaffected by the myoglobin. There was a slight difference between the two samples, with additional scattering visible between 0.01 Å-1-0.15 Å-1 in the hydrogel: myoglobin sample, which is consistent with the scattering of myoglobin.

Overall, the results demonstrate that the network morphology underpinning the hydrogel is not affected by the myoglobin and the data was consistent with a gel in which myoglobin molecules are loosely associated with the surface of the nanofibrils.

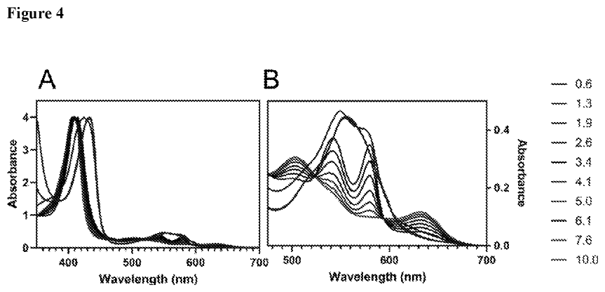

The effect of the gel association on the O₂-binding of myoglobin was investigated over 10 hours using UV-vis spectroscopy according to the procedure of Schenkman, 1997 (FIG. 2E). The hydrogel containing no myoglobin displayed limited absorption throughout the wavelength range. The reduced deoxymyoglobin spectra at t=0 exhibited a characteristically intense Soret band at 426 nm (Ghosh et al., 2014). The full transition from reduced deoxymyoglobin to oxymyoglobin to oxidized myoglobin (metmyoglobin), observable by an intense band at ˜409 nm and a weaker Q-band at 628 nm, took place over the course of 10 hours. The transition is significantly more rapid than for an identical protein preparation in identical conditions, other than the presence of the SAP hydrogel. The observation of those spectral changes, which are the same as those observed during the oxidation of normal myoglobin in solution (FIG. 4), demonstrate that myoglobin is incorporated within the hydrogel in a functional state. Interestingly, the rate of oxidation within the hydrogel was substantially slower than what was observed in the absence of the hydrogel, which suggests that the oxygen diffusion coefficient of the hydrogel is lower, which is consistent with previous studies of oxygen diffusion through hydrogels (White et al., 2014).

### Example 4. In Vivo Transplantation of Cells with Myoglobin

All animal procedures and methods were conducted in accordance with the Australian National Health and Medical Research Council's published Code of Practice for the Use of Animals in Research and were approved by the Florey Institute of Neuroscience and Mental Health Animal Ethics Committee. Cells for transplantation were obtained from time mated mice expressing green fluorescent protein (GFP) under the β-actin promoter, which enable clear distinction of the grafted cells (GFP+) cells within the host brain. Cortical brain tissue was isolated from pups at embryonic day 14.5 (E14.5), dissociated and resuspended at 100,000 cells/μl until the time of surgery. Hydrogels were sterilized by UV lamp for 2 hours and stock myoglobin was filtered by syringe filters (0.2 μm). Cells and gels were mixed at a 1:1 ratio immediately prior to in vivo delivery (Wang et al., 2012).

Adult C57BL/6 mice (n=6) were anaesthetized with 2% isoflurane and placed in the stereotaxic frame. A craniotomy was performed and unilateral microinjections of cells and hydrogels (total 2 μl) were implanted into the host striatum (0.5 mm anterior and 2 mm lateral to Bregma, and 3 mm below the surface of the brain). After 28 days, mice were killed by an overdose of sodium pentobarbitone (100 mg/kg) and transcardially perfused with warm saline followed by 4% paraformaldehyde (PFA). Brains were removed, post-fixed for two hours in 4% PFA and cryo-preserved overnight in 30% sucrose solution. Brains were sectioned on the coronal plane using a freezing microtome (40 μm thickness, 1:10 series).

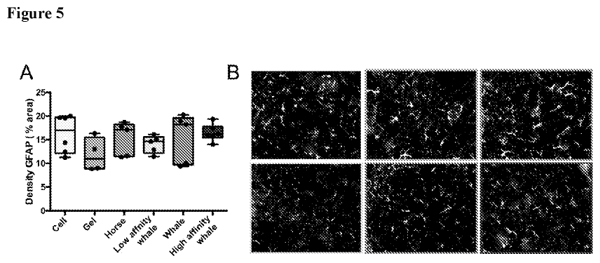

The biocompatibility of the hydrogel oxygen vectors within the host brain was next investigated to determine how myoglobin functionalisation and oxygen delivery impacted the host immune response. The resultant peptide/protein concentration of 10 mg mL-1 was administered through ultrafine glass capillaries into the brain. At 28 days post administration, neither the Fmoc-DDIKVAV peptide hydrogel, nor the hydrogel functionalised with myoglobin, showed any detrimental impact (i.e., no increase in local immune-responsive cells). This was determined through examination of the density of proinflammatory GFAP+ astrocytes immediately adjacent to the site of hydrogel administration within the brain, where no increase in the number of reactive astrocytes was observed (FIG. 5).

### Example 5. Immunohistochemistry & Quantification

Immunohistochemistry was performed on free-floating brain sections as previously described (Somaa et al., 2017). Brain sections were washed and incubated in primary antibodies overnight at room temperature, including: rabbit anti-GFP (1:20,000; Abcam), chicken anti-GFP (1:20,000; Abcam), sheep anti-Ki67 (1:40, R&D Systems), goat anti-doublecortin (DCX) (1:1000, Santa Cruz), chicken anti-GFAP (anti-glial fibrillary acidic protein, 1:500, Dako), mouse anti-NeuN (1:1000; Abcam). The following day sections were rinsed and blocked in 5% donkey serum for 20 minutes. Secondary antibodies for (i) direct detection were used at a dilution of 1:200—DyLight 488, 549 or 649 conjugated donkey anti-mouse, anti-sheep, anti-chicken or anti-rabbit (Jackson ImmunoResearch); and (ii) indirect with streptavidin-biotin amplification-biotin conjugated donkey anti-rabbit (1:500; Jackson ImmunoResearch) followed by peroxidase conjugated streptavidin (Vectastain ABC kit, Vector laboratories). Finally, fluorescently labelled sectioned were stained with 4′, 6-diamidino-2-phenylindole (DAPI, 1:5000, Sigma-Aldrich) to enable visualisation of all cells. Sections were mounted onto gelatinized slides and coverslipped. All fluorescent images were captured using a Zeiss Axio Observer.Z1 epifluorescence and bright images were obtained using a Leica DM6000 upright microscope.

Total number of NeuN, DAPI, Ki67 and DCX cells within the graft, as well as the density of NeuN+, Ki67+ and DCX+ cells in grafts were counted from images captured at 40× magnification and expressed as per mm³. The density of graft-derived GFP⁺ fibers as well as host-derived GFAP⁺ density (assessed as % immunoreactive pixels) were assessed at the graft-host border, as previously described and analysed by Nisbet et al., 2018.

Having established that the Mb-functionalised hydrogel is biocompatible, the effect of Mb on the survival and differentiation of transplanted progenitor cells as then investigated. Green fluorescent protein reporter (GFP+) neuronal progenitor cells were incorporated within the peptide hydrogel (+/−myoglobin) for co-delivery into the brain. The GFP reporter within the cells enables clear identification of transplanted cells and their neuronal processes within the host brain tissue. Using GFP to delineate the graft core, it was observed a significantly increased graft volume (p=0.029), when Mb was incorporated within the hydrogel vs. the no-Mb control (FIG. 5).

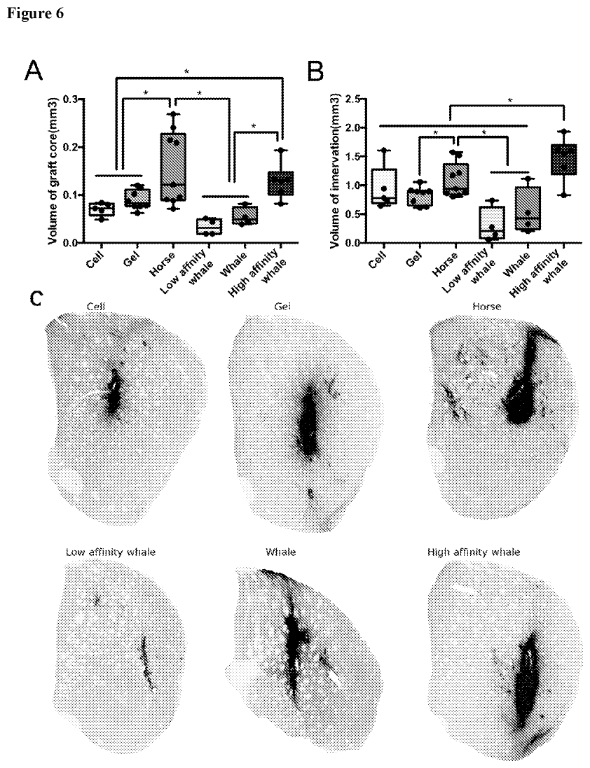

To investigate the effect of Mb on cell differentiation, the density of NeuN+ cells within the graft 28 days post transplantation was quantified. An IKVAV epitope-containing hydrogel scaffold has been previously shown to be capable of increasing the proportion of neurons within the graft because the high availability and surface density of the IKVAV epitope in the SAP promotes neuronal adhesion, differentiation and axonal growth of neural progenitors more efficiently than laminin itself (Nisbet et al., 2018; Rodriguez et al., 2018; Wang et al., 2020). That IKVAV epitope-containing hydrogel resulted in a density of neurons of 807±89 cells/mm³ within the graft core (FIG. 6). An additional significant increase (p=0.02) in neuronal differentiation to a density of 1205±92 cells/mm³ was observed as a result of the sustained delivery of oxygen via myoglobin using the hydrogel of the present invention (FIG. 6). As the base hydrogel structures are identical (FIG. 2), the increased differentiation using the hydrogel of the present invention suggests that the presence of myoglobin within the hydrogel can not only enhance the delivery and long-term survival of grafted cortical neural stem cells, but also bias their differentiation towards the neuronal fate that is critical for circuitry repair and reconstruction.

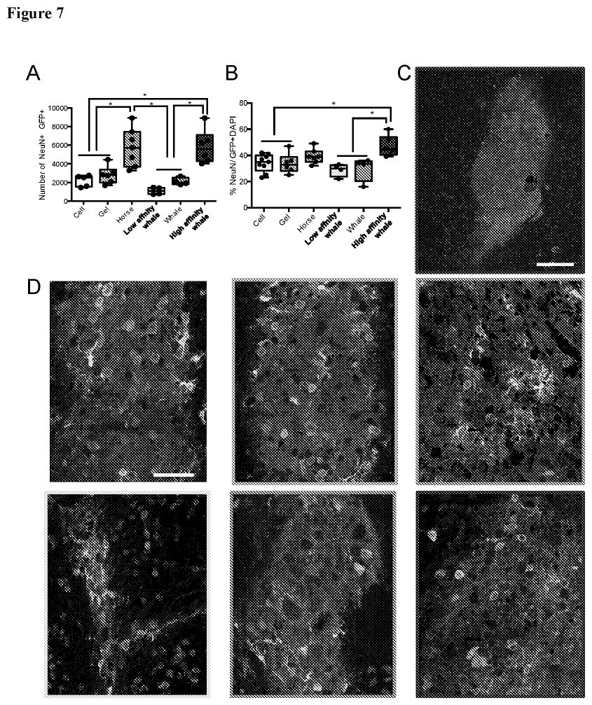

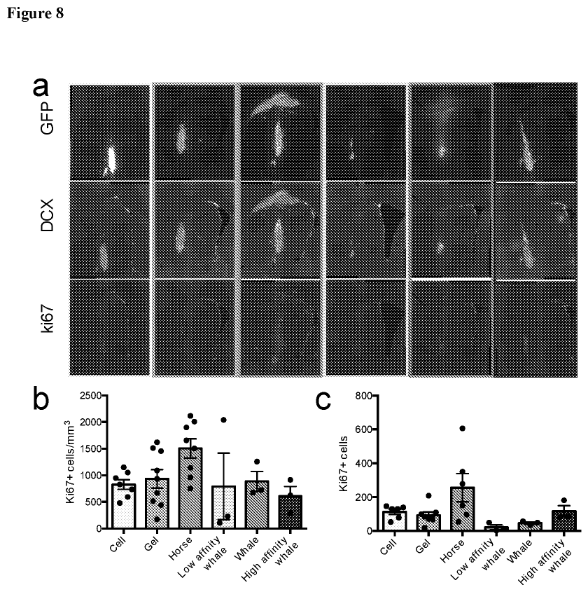

In light of the enhanced neuronal differentiation observed due to the presence of Mb in the SAP hydrogel, the ability of cells to integrate into the host brain was also investigated, as an indicator of brain repair. Graft integration was assessed at 28 days post administration by volumetric analysis of the GFP+ fibres within the surrounding brain parenchyma. The results showed that the transplanted GFP+ progenitors were capable of integrating into the host tissue, with extensive GFP+ fibre growth observed surrounding the graft core and innervating the host tissue (FIG. 7). Again, there was a statistically significant increase in innervation volume (p=0.0009) in the Mb: SAP hydrogel over the SAP hydrogel without Mb, increasing almost 2-fold from 3.3±0.4 mm³ to 6.1±0.5 mm³, suggesting axonal growth from cells delivered within the peptide hydrogel was not impeded post neuronal differentiation and that the delivery of oxygen within the graft core did not result in GFP+ fibres being restricted within the graft core. That is evidenced by the fact that the same GFP+ fibre density was observed within the parenchyma both with and without myoglobin functionalisation (FIG. 8). Furthermore, doublecortin (DCX+) migrating neuroblasts were observed at both the edge of the grafts and within the surrounding parenchyma, similarly demonstrating that the presence of the hydrogel scaffold (or formation of host scar tissue) did not impede grafted cell migration and integration within the host brain (FIG. 8). Therefore, myoglobin functionalisation of the hydrogel resulted in enhanced integration of neural stem cell grafts into existing host parenchyma.

The grafts were further assessed for evidence of excessive cell proliferation 28 days post implantation using the expression of Ki67 to mark cells undergoing proliferation according to the method of Miller et al., 2018. Excessive proliferation was not observed, with the density of Ki67+ cells being the same between the functionalised and unfunctionalized hydrogels (FIG. 8). Those Ki67+ cells are closely associated with blood vessels and may be endothelial progenitor cells undergoing angiogenesis within the graft tissue to form new blood vessels (Nisbet et al., 2018; Somaa et al., 2017).

Taken together, the in vivo data suggest the incorporation of myoglobin in a SAP hydrogel can promote the long-term survival and integration of stem cell transplants within the host brain, whilst avoiding undesirable cell fates and immune responses.

### Example 6. Control of Cell Survival and Differentiation Via Oxygen Homeostasis

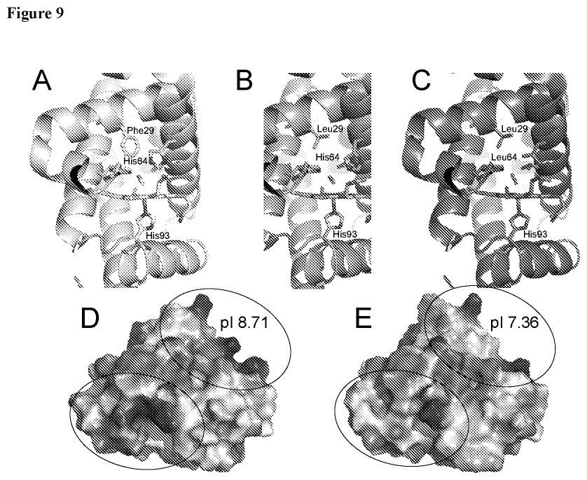

The parameters governing the beneficial role of oxygen affinity in supporting transplanted stem cell growth and differentiation were investigated using myoglobin variants. Wild-type sperm whale myoglobin (Physeter macrocephalus) was chosen, as well as the Leu29Phe mutant (which increases oxygen affinity by suppressing His64 fluctuations away from H-bonding with bound oxygen) and the His64Leu mutant (which has lower affinity for oxygen because the oxygen binding His64 sidechain is replaced with leucine) (FIG. 9). The Leu29Phe mutation increases oxygen affinity 13-fold from wild-type and the His64Leu mutation decreases oxygen affinity 55-fold from wild-type, altogether spanning an oxygen affinity (P50 mm Hg) of almost three orders of magnitude (0.007 to 53; 757-fold) (Dasmeh & Kepp, 2012; Scott et al., 2001). The low affinity variant is likely to release oxygen rapidly in physiological conditions, and the high affinity variant will not release bound oxygen until the environment becomes more hypoxic (low [O₂]) and would thus be expected to release O₂ more slowly.

Wild-type sperm whale myoglobin was mutated using site directed mutagenesis to generate the Leu29Phe and His64Leu mutants, and all three were heterologously expressed in Escherichia coli and purified to homogeneity. As those mutations were within the O₂ binding site in the interior of the protein, they were not expected to affect the properties of the gel, which was confirmed in the biophysical analysis of the gel (FIG. 2). There was no effect on the immune response from any of the sperm whale myoglobin hydrogels (FIG. 5), nor any increase in cell proliferation (FIG. 8). That suggested oxygen release (and the concentrations tested here) does not directly affect these processes.

However, when the growth, innervation and differentiation of the transplanted cells were examined, statistically significant differences were observed across the series of low to high affinity Mb. Both the volume of the graft core (3.8-fold) and the level of innervation (4.8-fold) increased significantly from His64Leu, to the Leu29Phe variant (FIG. 6). Likewise, the number of neural cells and level of cell differentiation increased significantly from the low affinity His64Leu variant to the Leu29Phe mutant (FIG. 7). The results demonstrate not only that Mb contributes to improved graft survival owing to its ability to bind and release oxygen in hypoxic conditions, but the levels of oxygen binding and release (engineered homeostasis) can be mediated by the selection of a Mb variants to tune the reservoir of oxygen to the demands of the transplanted cells.

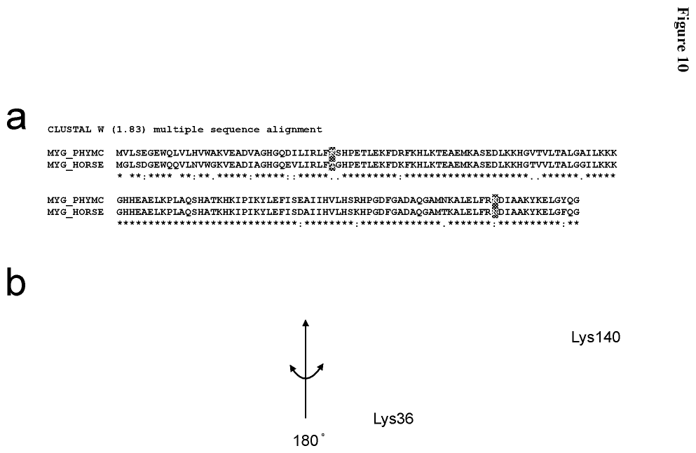

The effect of surface charge (measured as pI; isoelectric point) on the performance of Mbs in the gel was also tested. Wild-type horse and sperm whale myoglobin were selected for comparison because they have almost identical oxygen affinity, and very similar sequences (88% amino acid identity and 99% similarity) (FIG. 10), yet their surface charge differs (FIG. 9). FIG. 10 shows there are only 12 amino acid differences between the sequences, most of which are conservative. There are two differences that make the MYGPHYMC more positively charged: Lys34Thr and Lys 140Asn (blue), and 4 differences between polar and hydrophobic sidechains Leu10Gln, Ser35Gly, Val66Thr, Thr67Val, which are balanced between the sequences (2 polar>nonpolar vs. 2 nonpolar>polar). All mutations are surface exposed (red main chain atoms). The two sequence differences that contribute to higher positive surface charge (Lys36, Lys140) are shown in blue (main chain spheres and side chain sticks).

Mbs from deep diving animals (such as whales) are thought to have evolved high cationic surface charge for electrostatic repulsion to protect against aggregation owing to high concentration of myoglobin in their muscle tissue (Isogai, 2018). Indeed, sperm whale myoglobin has a significantly higher pI than horse (8.71 vs 7.36; FIG. 9). As can be seen in FIGS. 6 and 7, when only the wild-type Mb are compared, Horse Mb exhibits superior performance compared to wild-type sperm whale Mb in terms of cell survival and differentiation. That suggests the higher, cationic, surface charge of horse myoglobin, could result in slightly lower affinity to the hydrogel and leaching from the hydrogel, given the peptide: protein interaction will rely on hydrophobic and potentially anionic interactions with the exposed IKVAV motif.

### Example 7. Stability of Myoglobin in Hydrogel

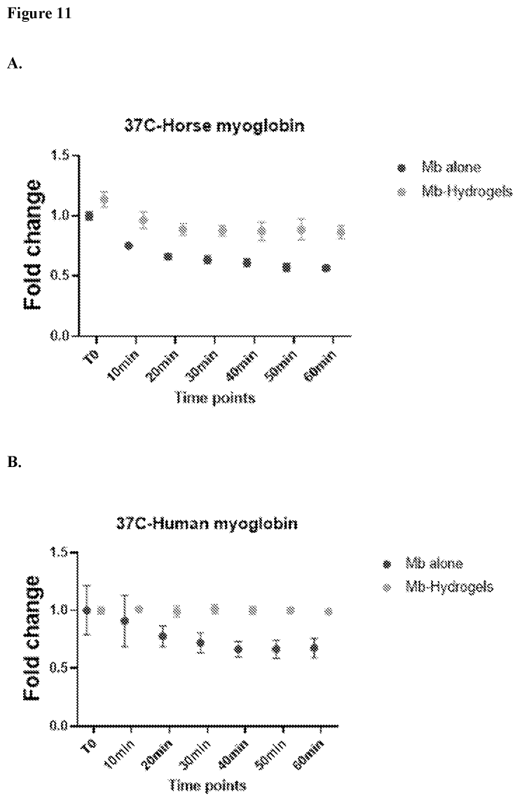

The stability of myoglobin (Mb) in hydrogels was assessed according to the procedure outlined by Z. Yang et al. (2020), the contents of which are incorporated herein by reference in their entirety, with some modifications. Briefly, horse and human wild type Mb were first dissolved to a concentration of 20 mg/mL in PBS. 50 μl aliquots were further dissolved to a final concentration of 1 mg/mL on either PBS hydrogel or on a 1:1 ratio of Fmoc-ddikvav: Fmoc-frgdf peptide hydrogel mix. Briefly, Fmoc-ddikvav was prepared as per Example 2 above and Fmoc-frgdf (15 mg/mL) was prepared by adding 400 μl of water to 10 mg of fmoc-frgdf, followed by 40 μl of NaOH (0.5M). 0.1 M of hydrochloric acid (HCl) was then added dropwise with continuous vortexing until the solution reached physiological relevant pH. Immediately after preparation, 100 μl was per sample was transferred using triplicates per group in a 96 well plate (flat bottom). The absorbance reading was performed on a CLARIOSTAR (BMG LABTECH) plate reader measuring absorbance spectrums (scan resolution 1 nm, 300-800 nm) every 10 min up to 2 h. The test was performed at controlled temperature and CO₂ conditions using an atmospheric control unit (BMG LABTECH), setting 37° C. and 5% CO₂. The results based on the optical density (O.D) at 410 nm, blank corrected for PBS or peptides alone, were normalized by time 0 and plotted as mean and standard deviation (FIG. 11). Statistical analysis was performed using two-way ANOVA (p>0.05).

The fold change of Mb at 410 nm has been previously reported and has been associated with the denaturation of the protein over time. In contrast, and as shown in FIG. 11, Mb entrapped in hydrogels showed sustained absorbance values, suggesting non-denaturation of the protein. These data show that the hydrogels provided a protective effect against Mb denaturation. A significant statistical difference (by 2-way ANOVA) was found for Mb alone vs Mb-Hydrogels at 20 min, 30 min and 60 min for horse Mb (FIG. 11A) and at 40 min for human Mb (FIG. 11B) (p<0.05).

### Example 8. Transplantation of Human Neural Precursors with Hydrogels into the Gut

Human induced pluripotent stem cells (iPSC)-derived embryonic neural progenitor cells (iENPs) were transduced with AAV-PHP.S-tdTomato. Wild-type and Ednrb knockout rats (a model of Hirschsprung disease, as described by Furness et al. (2023) and Stamp et al. (2022), the entire contents of which is incorporated herein by reference) were immunosuppressed using Cyclosporine for 7 days prior to cell implantation. For cell implantation, cell suspensions were prepared using 15 mg/ml IKVAV (a laminin fragment; Ile-Lys-Val-Ala-Val) hydrogel with 10 μm fluorescent (“blue”; ˜405 nm) beads (Thermo Fisher). The hydrogel/beads mix was vortexed at mid speed for 5 seconds and then pipetted up and down 10 times. The cells (1×10⁶) were then added to the hydrogel/beads mix and pipetted to mix. The cell-hydrogel suspensions were then implanted intramuscularly into the wall of the mid-distal colon using ˜100 μm bevelled pulled-glass electrodes attached to a Hamilton syringe. Animals were immunosuppressed with Cyclosporine for a further 7 days after cell implants. Animals were then killed and the mid-distal colon was dissected, stretched and pined in a Sylgard lined petri dish. The tissue was then fixed with 4% paraformaldehyde (PFA) overnight at 4° C. followed by 3×10-minute PBS washes. Colon samples were then separated into muscle (myenteric) and submucosal preparations. Submucosal preparations were then incubated in PBS with 1% Triton for 2 hours at room temperature followed by incubation in primary antibody (MHCII for macrophages, RFP to enhance endogenous td-Tomato and Hu for neurons) for 48 hours at 4° C. and then secondary antibody for 48 hours at 4° C. Images were acquired using a LSM800 confocal microscope.

The results showed that, when suspensions of human pluripotent stem cell-derived enteric neural precursors, combined with IKVAV hydrogels, were transplanted into the muscle wall of the distal colon of recipient rats, the cells survived and developed neuron-like morphologies. Relevantly, the hydrogels appeared to (i) keep the transplanted cells at the injection site more contained/localised, (ii) supported the survival of implanted enteric neural precursors and (iii) prevented the infiltration of antigen presenting cells at the injection site.

### REFERENCES

Bertani, G. Studies on lysogenesis I: the mode of phage liberation by lysogenic Escherichia coli. Journal of bacteriology 62, 293-300 (1951).
Bjellqvist, B. et al. The focusing positions of polypeptides in immobilized pH gradients can be predicted from their amino acid sequences. Electrophoresis 14, 1023-1031 (1993).
Burke, M. et al. Regulation of Scaffold Cell Adhesion Using Artificial Membrane Binding Proteins. Macromolecular Bioscience 17, 1600523 (2017).
Casaccia-Bonnefil, P. Cell death in the oligodendrocyte lineage: A molecular perspective of life/death decisions in development and disease. Glia 29, 124-135 (2000).
Castro-Forero, A., Jimenez, D., Lopez-Garriga, J. & Torres-Lugo, M. Immobilization of myoglobin from horse skeletal muscle in hydrophilic polymer networks. J Appl Polym Sci 107, 881-890 (2008).
Dasmeh, P. & Kepp, K. P. Bridging the gap between chemistry, physiology, and evolution: quantifying the functionality of sperm whale myoglobin mutants. Comparative Biochemistry and Physiology Part A: Molecular & Integrative Physiology 161, 9-17 (2012).
Di Tommaso, P. et al. T-Coffee: a web server for the multiple sequence alignment of protein and RNA sequences using structural information and homology extension. Nucleic acids research 39, W13-W17 (2011).
Doherty, J. & Baehrecke, E. H. Life, death and autophagy. Nat Cell Biol 20, 1110-1117 (2018).
Furness J. B. et al. Development of the aganglionic colon following surgical rescue in a cell therapy model of Hirschsprung disease in rat. Dis Model Mech. 2023: dmm.050055. doi: 10.1242/dmm.050055 (2023).
Ghosh, K., Kumar, R., Kumar, K., Ratnam, A. & Singh, U. P. Reactivity of nitric oxide with ruthenium complexes derived from bidentate ligands: structure of a ruthenium nitrosyl complex, photoinduced generation and estimation of nitric oxide. Rsc Adv 4, 43599-43605 (2014).
Gibson, D. G. et al. Enzymatic assembly of DNA molecules up to several hundred kilobases. Nature methods 6, 343-345 (2009).
Horgan, C. C. et a Characterisation of minimalist co-assembled fluorenylmethyloxycarbonyl self-assembling peptide systems for presentation of multiple bioactive peptides. Acta Biomater 38, 11-22 (2016).
Isogai, Y. et al. Tracing whale myoglobin evolution by resurrecting ancient proteins. Scientific reports 8, 1-14 (2018).
Maclean, F. L., Ims, G. M., Horne, M. K., Williams, R. J. & Nisbet, D. R. A Programmed Anti-Inflammatory Nanoscaffold (PAIN) as a 3D Tool to Understand the Brain Injury Response. Adv Mater 30 (2018).
Miller, I. et al. Ki67 is a graded rather than a binary marker of proliferation versus quiescence. Cell reports 24, 1105-1112. e1105 (2018).
Nagao, S. et al. Structural and oxygen binding properties of dimeric horse myoglobin. Dalton Transactions 41, 11378-11385 (2012).
Nisbet, D. et al. Shear containment of BDNF within molecular hydrogels promotes human stem cell engraftment and postinfarction remodeling in stroke. Advanced Biosystems 2, 1800113 (2018).
Rodriguez, A. L., Parish, C. L., Nisbet, D. R. & Williams, R. J. Tuning the amino acid sequence of minimalist peptides to present biological signals via charge neutralised self assembly. Soft matter 9, 3915-3919 (2013).
Rodriguez, A. L. et al. Using minimalist self-assembling peptides as hierarchical scaffolds to stabilise growth factors and promote stem cell integration in the injured brain. J Tissue Eng Regen M 12, E1571-E1579 (2018).
Schenkman, K. A., Marble, D. R., Burns, D. H. & Feigl, E. O. Myoglobin oxygen dissociation by multiwavelength spectroscopy. Journal of Applied Physiology 82, 86-92 (1997).
Scott, E. E., Gibson, Q. H. & Olson, J. S. Mapping the pathways for O2 entry into and exit from myoglobin. Journal of Biological Chemistry 276, 5177-5188 (2001).
Somaa, F. A. et al. Peptide-based scaffolds support human cortical progenitor graft integration to reduce atrophy and promote functional repair in a model of stroke. Cell reports 20, 1964-1977 (2017).
Springer, B. A. & Sligar, S. G. High-level expression of sperm whale myoglobin in Escherichia coli. Proceedings of the National Academy of Sciences 84, 8961-8965 (1987).
Stamp L. A. et al. Surgical method to prevent early death of neonatal rat pups with Hirschsprung disease, thus permitting development of long-term therapeutic approaches. Biology Methods and Protocols, 7 (1), bpac004 (2022).
Wang, T.-Y., Forsythe, J. S., Nisbet, D. R. & Parish, C. L. Promoting engraftment of transplanted neural stem cells/progenitors using biofunctionalised electrospun scaffolds. Biomaterials 33, 9188-9197 (2012).
Wang, Y. et al. Peptide Programmed Hydrogels as Safe Sanctuary Microenvironments for Cell Transplantation. Advanced Functional Materials 30, 1900390 (2020).
White, J. C., Godsey, M. E. & Bhatia, S. R. Perfluorocarbons enhance oxygen transport in alginate-based hydrogels. Polymers for advanced technologies 25, 1242-1246 (2014).
Z. Yang et al. Targeted Myoglobin Delivery as a Strategy for Enhancing the Sensitivity of Hypoxic Cancer Cells to Radiation, CellPress 23 (6), 101158 (2020).

1. A biocompatible hydrogel for delivering a cell into a subject, the hydrogel comprising a scaffold and an oxygen carrier non-covalently bound to the scaffold.

2. The biocompatible hydrogel of claim 1, which is a self-assembled hydrogel.

3. The biocompatible hydrogel of claim 1, which is an injectable hydrogel.

4. The biocompatible hydrogel of claim 1, wherein the scaffold is a peptide-based scaffold.

5. The biocompatible hydrogel of claim 1, wherein the peptide-based scaffold comprises an extracellular matrix (ECM) protein or a functional variant thereof.

6. The biocompatible hydrogel of claim 5, wherein the functional variant comprises a binding domain of the ECM protein.

7. The biocompatible hydrogel of claim 5, wherein the ECM protein is selected from the group consisting of laminin, collagen, fibrin, fibronectin, gelatin, elastin, hyaluronan, a proteoglycan, a polysaccharide, an enzyme, an integrin, a functional variant of any of the foregoing, and any combination thereof.

8. The biocompatible hydrogel of claim 7, wherein the ECM protein is laminin or a functional variant thereof.

9. The biocompatible hydrogel of claim 4, wherein the scaffold comprises Fmoc-DDIKVAV or Fmoc-DDIKVAVD.

10. The biocompatible hydrogel of claim 1, wherein the oxygen carrier is an oxygen carrier protein.

11. The biocompatible hydrogel of claim 10, wherein the oxygen carrier is selected from the group consisting of myoglobin, haemoglobin, neuroglobin, cytoglobin, a functional variant of any of the foregoing, and any combination thereof.

12. The biocompatible hydrogel of claim 11, wherein the oxygen carrier protein is myoglobin or a functional variant thereof.

13. The biocompatible hydrogel of claim 12, wherein the functional variant of myoglobin is a Leu29Phe or His64Leu mutant.

14. The biocompatible hydrogel of claim 1, wherein the oxygen carrier is electrostatically bound to the scaffold.

15. The biocompatible hydrogel of claim 1, further comprising a cell.

16. The biocompatible hydrogel of claim 15, wherein the cell is a stem cell.

17. An injectable self-assembled The biocompatible hydrogel of claim 1 comprising an injectable self-assembled biocompatible hydrogel comprising an IKVAV-based scaffold and myoglobin, or a functional variant thereof, non-covalently bound to the scaffold.

18. (canceled)

19. A method for preparing the biocompatible hydrogel of claim 1, the method comprising:
a1) (i) combining a scaffold material and water to form a hydrogel scaffold; and
(ii) combining the hydrogel scaffold with an oxygen carrier to form a homogenous mixture;
or
a2) combining a scaffold material, an oxygen carrier and water to form a homogenous mixture;
and
b) gelation of the homogenous mixture to form a biocompatible hydrogel in which the oxygen carrier is non-covalently bound to the hydrogel scaffold.

20. (canceled)

21. (canceled)

22. (canceled)

23. The method of claim 19, further comprising incorporating cells into the biocompatible hydrogel.

24. (canceled)

25. A method for transplanting a cell into a subject, the method comprising administering a cell and the biocompatible hydrogel of claim 1 to a tissue of the subject.

26. (canceled)

27. (canceled)

28. (canceled)

29. (canceled)

30. The method of claim 25, for the treatment of Parkinson's disease.

31. (canceled)

32. (canceled)


SEQUENCE LISTING (4 sequences)

SEQ ID NO: 1 - DNA, 52 bp, Physeter macrocephalus
  source 1..52: mol_type = other DNA
gtcgctggtcatggtcaggacatcttcattcgactgttcaaatctcatccgg

SEQ ID NO: 2 - DNA, 52 bp, Physeter macrocephalus
  source 1..52: mol_type = other DNA
cggatgagatttgaacagtcgaatgaagatgtcctgaccatgaccagcgacg

SEQ ID NO: 3 - DNA, 52 bp, Physeter macrocephalus
  source 1..52: mol_type = other DNA
gaaagcttctgaagatctgaaaaaactgggtgttaccgtgttaactgcccta

SEQ ID NO: 4 - DNA, 52 bp, Physeter macrocephalus
  source 1..52: mol_type = other DNA
tagggcagttaacacggtaacacccagttttttcagatcttcagaagctttc